(19) United States
(12) Patent Application Publication
(10) Pub. No.: US 2026/0295056 A1
(43) Pub. Date: Oct. 1, 2026

(54) LYMPH NODE METAL THERAPY FOR THE TREATMENT OF CANCER AND IMMUNE-DEFICIENCY RELATED DISEASE

(71) Applicants: INSTITUT CURIE, PARIS (FR); INSTITUT NATIONAL DE LA SANTÉ ET DE LA RECHERCHE MÉDICALE, PARIS (FR); CENTRE NATIONAL DE LA RECHERCHE SCIENTIFIQUE, PARIS (FR); PRESIDENT AND FELLOWS OF HARVARD COLLEGE, CAMBRIDGE, MA (US)

(72) Inventors: RAPHAËL RODRIGUEZ, PARIS (FR); STÉPHANIE SOLIER, PARIS (FR); SÉBASTIAN MÜLLER, IVRY-SUR-SEINE (FR); TERESA TATIANA CAÑEQUE, PARIS (FR); JESSALYN UBELLACKER, QUINCY, MA (US)

(21) Appl. No.: 19/477,888

(22) PCT Filed: Apr. 25, 2024
(86) PCT No.: PCT/EP2024/061372

§ 371 Date: Oct. 23, 2025

(30) Foreign Application Priority Data
Apr. 26, 2023 (EP) 23170172.3

(51) Int. Cl. A61K 47/61; A61K 33/26; A61K 33/34; A61K 45/06; A61K 47/36; A61P 35/00

(52) U.S. Cl. CPC A61K 47/61; A61K 33/26; A61K 33/34; A61K 45/06; A61K 47/36; A61P 35/00

(57) ABSTRACT

The invention relates to a pharmaceutical composition for use in a method for the treatment of cancer and/or immune deficiency diseases and/or infectious diseases, comprising a metal ion and optionally hyaluronate (HA) or a hyaluronate conjugate, wherein said method comprises the intralymphatic administration of the pharmaceutical composition to a subject. The invention also relates to a pharmaceutical combination for use in a method for the treatment of cancer, the pharmaceutical combination comprising: (i) at least one metal ion, preferably selected in the group consisting of iron (Fe), copper (Cu), manganese (Mn), calcium (Ca), magnesium (Mg), zinc (Zn), and mixtures thereof; and (ii) hyaluronate (HA) or a hyaluronate conjugate, wherein said method comprises the simultaneous, separate or sequential administration of (i) and (ii) directly into the lymphatic system, preferably into a lymph node or a lymph vessel.

### FIELD OF THE INVENTION

The invention relates to the treatment of cancer and immune-deficiency related disease. In particular, the invention thus relates to the direct administration of metal ion(s), hyaluronate (HA), hyaluronate conjugate, or combination thereof into the lymphatic system, to enable immune cells in the nodes to be activated.

### BACKGROUND OF THE INVENTION

Cancer is considered mostly incurable. While standard-of-care can, in some cases exhibit beneficial therapeutic effects, cancer in most cases relapse. This is partly the result of ineffective immune system and the lack of drug targeting metastasis.

In the case of cancer metastasis, circulating tumor cells migrate to the lymphatic system, in particular to the lymph nodes, where activation is meant to take place. Indeed, circulating tumor cells colonize the lymphatic system to prime cancer cells and initiate an immune response. If successful, this reverses tumor growth. If this fails, the primary tumor develops, and metastasis occur in the lymph node and other tissues.

There is thus a need to find new method to promote activation of immune cells in the lymphatic system, in particular for the treatment of cancer and immune-deficiency related disease.

### SUMMARY OF THE INVENTION

The present invention consists of supplying the immune system with the required chemicals to promote activation of immune cells.

In particular, the inventors have shown that immune cells (e.g. T cells, macrophages, dendritic cells . . . ) use CD44-mediated iron and copper uptake to activate genes involved in the immune responses. However, in cancer, immune cell activation takes place in the lymphatic system (e.g. lymph nodes), and the inventors have shown that lymphatic system is naturally deprived in metals, hyaluronates, which is the main ligand of CD44, presumably to prevent permanent immune cell activation and excessive responses. In particular, the lymphatic system is deprived of iron and copper.

The invention thus relates to the use of metals and/or hyaluronates supplemented directly in the lymphatic system to avoid loading the blood and feeding the tumor with more metals (including, iron, copper, manganese and calcium). Hyaluronates and metals (iron, copper, manganese) is expected to boost immune cell activation and anti-cancer activity when the treatment is supplied directly to the lymphatic system, in particular via the lymph node.

In other words, the invention thus relates to the direct administration of metal ion(s), hyaluronate (HA), hyaluronate conjugate, or combination thereof into the lymphatic system, to enable immune cells in the nodes to be activated.

One aspect of the invention relates to a pharmaceutical composition for use in a method for the treatment of cancer and/or immune deficiency diseases, comprising a metal ion or a combination of a metal ion with hyaluronate (HA) or a hyaluronate conjugate, wherein said method comprises the intralymphatic administration of the pharmaceutical composition to a subject.

Another aspect of the invention relates to a complex of hyaluronate or hyaluronate conjugate and at least one metal ion, wherein said hyaluronate presents a molecular weight between 1-10 000 kDa and said metal ion is selected in the group consisting of iron (Fe), copper (Cu), manganese (Mn), calcium (Ca), magnesium (Mg) and zinc (Zn).

Another aspect of the invention relates to a complex of hyaluronate or hyaluronate conjugate and at least one metal ion, wherein said hyaluronate presents a molecular weight between 1 kDa-10 000 kDa, preferably between 600 kDa and 1000 kDa and said metal ion is selected in the group consisting of iron (Fe), copper (Cu), manganese (Mn), calcium (Ca), magnesium (Mg), zinc (Zn) and mixtures thereof.

Another aspect of the invention relates to a pharmaceutical composition comprising the complex according to the invention and a pharmaceutically acceptable excipient and/or carrier.

Another aspect of the invention relates to the complex according to the invention or a pharmaceutical composition as defined above, for use as a drug, in particular for use in a method for the treatment of cancer and/or immune deficiency diseases, preferably said method comprising the intralymphatic administration of the pharmaceutical composition to a subject.

Another aspect of the invention relates to a pharmaceutical combination for use in a method for the treatment of cancer and/or immune deficiency diseases, the pharmaceutical combination comprising:
(i) at least one metal ion, preferably selected in the group consisting of iron (Fe), copper (Cu), manganese (Mn), calcium (Ca), magnesium (Mg) and zinc (Zn); and
(ii) hyaluronate (HA) or a hyaluronate conjugate,
   wherein said method comprises the simultaneous, separate or sequential administration of (i) and (ii) directly into the lymphatic system, preferably into a lymph node or a lymph vessel.

### BRIEF DESCRIPTION OF THE FIGURES

FIG. 1 shows metal quantification in blood, serum and lymph from mice using inductively-coupled plasma mass spectrometry. N=6-7.

FIG. 2 shows metal quantification in the lymph nodes and surrounded tissues from mice. N=6.

FIG. 3 shows A) Metal quantifications in non-activated dendritic cells (naDC) and activated dendritic cells (DC) by ICP-MS. B) Metal quantifications in aDC in wild type (CD44+) and CD44 knockout conditions by ICP-MS.

FIG. 4 shows A) Metal quantifications in non-activated macrophages (naMDM) and activated macrophages (aMDM) by ICP-MS. B) Metal quantifications in aMDM in small-interference RNA control (siCtrl.) and CD44 knockdown conditions by ICP-MS.

FIG. 5 shows Metal quantification in non-activated CD4+ T cells (naCD4+) and activated CD4+ T cells (aCD4+) and in non-activated CD8+ T cells (naCD8+) and activated CD8+ T cells (aCD8+) with ICP-MS.

FIG. 6 shows Metal quantification in cell culture medium and fetal bovine serum (FBS) used for culture of immune cells in vitro.

FIG. 7 shows A) Metal quantification in aMDM using ICP-MS. a, control cells (ctrl.) and cells treated with a blocking CD44-antibody RG7356. B) control cells and cells treated with hyaluronic acid (HA) which can bind metal ions- or a methylated (meth.) HA-which cannot bind metal ions.

FIG. 8 shows flow cytometry of immune infiltrates of intra-lymphatic tumors in mice treated with vehicle or hyaluronate-metal complexes intra-lymphatically.

### DETAILED DESCRIPTION OF THE INVENTION

### Definitions

For the purpose of the invention, the term “pharmaceutically acceptable” is intended to mean what is useful to the preparation of a pharmaceutical composition, and what is generally safe and nontoxic, for a pharmaceutical use.

As used herein, the term “pharmaceutically acceptable excipient” refers to any ingredient except active ingredients which are present in a pharmaceutical composition. Its addition may be aimed to confer a particular consistency or other physical or gustative properties to the final product. A pharmaceutically acceptable excipient must be devoid of any interaction, in particular chemical, with the active ingredients.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheep and non-human primates, among others.

Within the context of the present disclosure, the term treatment denotes curative, symptomatic, and preventive treatment. Pharmaceutical compositions, kits, products and combined preparations of the invention can be used in humans with a disease or disorder. The pharmaceutical compositions, kits, products and combined preparations of the invention will not necessarily cure the patient but will delay or slow the progression or prevent further progression of the disease or disorder, and/or ameliorating thereby the patients' condition. In treating the disease or disorder, the pharmaceutical composition of the invention is administered in a therapeutically effective amount.

Whenever within this whole specification “treatment of a disease or disorder” or the like is mentioned with reference to the pharmaceutical composition of the invention, there is meant: a) a method for treating a disease or disorder, said method comprising administering a therapeutically effective amount of a compound of the invention or of a pharmaceutical composition comprising said compound to a subject in need of such treatment; b) the use of a compound of the invention or of a pharmaceutical composition comprising said compound for the treatment of a disease or disorder; c) the use of a compound of the invention or of a pharmaceutical composition comprising said compound for the manufacture of a medicament for the treatment of a disease or disorder; and/or d) a compound of the invention or of a pharmaceutical composition comprising said compound for use in the treatment a disease or disorder.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

By “therapeutically effective amount” or “active amount” or “effective amount”, it is meant the quantity of the pharmaceutical composition of the invention which prevents, removes or reduces the deleterious effects of a disease or disorder in mammals, including humans, alone or in combination with the other active ingredients of the pharmaceutical composition, kit, product or combined preparation. It is understood that the administered dose may be lower for each compound in the composition to the “therapeutic effective amount” define for each compound used alone or in combination with other treatments than the combination described here. The “therapeutic effective amount” of the composition will be adapted by those skilled in the art according to the patient, the pathology, the mode of administration, etc.

In the context of the invention, the ranges of values expressed by “from X to XX” or “between X and XX” comprise the upper and lower limits.

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus. The term “cancer” according to the invention comprises leukemias, seminomas, melanomas, teratomas, lymphomas, non-Hodgkin lymphoma, neuroblastomas, gliomas, adenocarcinoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Karposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Karposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochrondroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

Optionally, the cancer can be selected in the group consisting of rectal cancer, colorectal cancer, stomach cancer, head and neck cancer, thyroid cancer, cervical cancer, uterine cancer, breast cancer, in particular triple negative breast cancer, ovarian cancer, brain cancer, in particular glioblastoma and neuroblastoma, lung cancer, in particular small-cell lung cancer and non-small-cell lung cancer, skin cancer, bladder cancer, blood cancer, renal cancer, liver cancer, prostate cancer, multiple myeloma, pancreatic cancer and endometrial cancer.

The term “cancer” according to the invention preferably comprises solid cancer (including breast cancer, ovarian cancer, pancreatic cancer, lung cancer, colon cancer, prostate cancer, melanoma, liver cancer, and brain cancer), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

The term “immune deficiency diseases” or “autoimmune disease” relates to any disease that may affect or disrupt the immune system. The term “immune deficiency diseases” according to the invention comprises, but is not limited to, Autoimmune lymphoproliferative syndrome (ALPS), Autoimmune polyglandular syndrome type 1 (APS-1), BENTA disease, Caspase eight deficiency state (CEDS), Chronic Granulomatous Disease (CGD), Common Variable Immunodeficiency (CVID), Congenital Neutropenia Syndromes, CTLA4 deficiency, DOCK8 deficiency, GATA2 deficiency, Glycosylation Disorders with Immunodeficiency, Hyper-Immunoglobulin E Syndromes (HIES), Hyper-immunoglobulin M (IgM) syndromes, Interferon gamma, interleukin 12 and interleukin 23 deficiencies, Leukocyte adhesion deficiency (LAD), LRBA deficiency, PI3 Kinase Disease, PLCG2-associated Antibody Deficiency and Immune Dysregulation (PLAID), Severe Combined Immunodeficiency (SCID), STAT3 dominant-negative disease (STAT3DN), STAT3 gain-of-function disease, Warts, Hypogammaglobulinemia, Infections, and Myelokathexis (WHIM) Syndrome, Wiskott-Aldrich syndrome (WAS), X-Linked Agammaglobulinemia (XLA), X-Linked Lymphoproliferative Disease (XLP), XMEN disease.

In the context of the invention, “infectious diseases”, relates to any diseases related to the invasion of tissues by pathogens. The term “infectious diseases” according to the invention comprises, but is not limited to, viral diseases, bacterial diseases, or fungi or parasite related diseases.

In the context of the invention, the expression “intralymphatic administration” denotes the direct injection of a substance or composition into the lymphatic system. In a particular embodiment, intralymphatic administration is selected from injection into a lymph vessel or into a lymph node, preferably a lymph node.

### Description

In a first aspect, the invention relates to a pharmaceutical composition for use in a method for the treatment of cancer and/or immune deficiency diseases and/or infectious diseases, comprising a metal ion and optionally hyaluronate (HA) or a hyaluronate conjugate, wherein said method comprises the intralymphatic administration of the pharmaceutical composition to a subject.

In a specific embodiment, the pharmaceutical composition comprises a metal ion, preferably selected in the group consisting of iron (Fe), copper (Cu), manganese (Mn), calcium (Ca), magnesium (Mg) and zinc (Zn).

In a specific embodiment, the pharmaceutical composition comprises a metal ion, preferably selected in the group consisting of iron (Fe), copper (Cu), manganese (Mn), calcium (Ca), magnesium (Mg), zinc (Zn), and mixtures thereof. For example, the pharmaceutical composition can comprise a mixture of at least two or three metal ions selected in the group consisting of iron (Fe), copper (Cu), manganese (Mn), calcium (Ca), magnesium (Mg), and zinc (Zn). Typically, the pharmaceutical composition can comprise Fe and/or Cu, and at least one other metal ions selected in the group consisting of manganese (Mn), calcium (Ca), magnesium (Mg), and zinc (Zn). In another embodiment of the invention, the pharmaceutical composition comprises a metal ion and hyaluronate (HA).

In another embodiment of the invention, the pharmaceutical composition comprises a metal ion and hyaluronate conjugate.

In another embodiment of the invention, the pharmaceutical composition comprises a complex of the metal ion and hyaluronate (HA) or a hyaluronate conjugate.

The invention further relates to a complex of hyaluronate or hyaluronate conjugate and at least one metal ion, wherein said hyaluronate presents a molecular weight between 1-10 000 kDa and said metal ion is selected in the group consisting of iron (Fe), copper (Cu), manganese (Mn), calcium (Ca), magnesium (Mg) and zinc (Zn).

The invention further relates to a complex of hyaluronate or hyaluronate conjugate and at least one metal ion, wherein said hyaluronate presents a molecular weight between 1 kDa-10 000 kDa, preferably between 600 kDa and 1000 kDa and said metal ion is selected in the group consisting of iron (Fe), copper (Cu), manganese (Mn), calcium (Ca), magnesium (Mg), zinc (Zn) and mixtures thereof. The invention further relates to a pharmaceutical composition comprising the complex according to the invention and a pharmaceutically acceptable excipient and/or carrier.

The invention further relates to the complex according to the invention or the pharmaceutical composition comprising said complex, for use as a drug.

The invention further relates to the complex according to the invention or the pharmaceutical composition comprising said complex, for use in a method for the treatment of cancer and/or immune deficiency diseases and/or infectious diseases, preferably said method comprising the intralymphatic administration of the pharmaceutical composition to a subject.

The invention further relates to a pharmaceutical combination or a combination product for use in a method for the treatment of cancer and/or immune deficiency diseases and/or infectious diseases, the pharmaceutical combination or the combination product comprising:
(i) at least one metal ion, preferably selected in the group consisting of iron (Fe), copper (Cu), manganese (Mn), calcium (Ca), magnesium (Mg), zinc (Zn), and mixtures thereof; and
(ii) hyaluronate (HA) or a hyaluronate conjugate,
   wherein said method comprises the simultaneous, separate or sequential administration of (i) and (ii) directly into the lymphatic system, preferably into a lymph node or a lymph vessel.

In a particular embodiment of the invention, the pharmaceutical combination or the combination product is a pharmaceutical composition comprising (i), (ii), and a pharmaceutically acceptable carrier.

In another embodiment of the invention, the pharmaceutical combination or the combination product is a kit of parts comprising (i) and (ii).

In all these embodiments of the invention, the method for the treatment of cancer and/or immune deficiency diseases and/or infectious diseases, preferably comprises the injection of the pharmaceutical composition of the invention or the complex of the invention or the pharmaceutical combination, into the lymphatic system, preferably into a lymph vessel or a lymph node.

In all these embodiments of the invention, the metal ion is preferably a metal cation. Advantageously, the metal ion is selected in the group consisting of iron (Fe), copper (Cu), manganese (Mn), calcium (Ca), magnesium (Mg), zinc (Zn), and mixtures thereof. Advantageously, the metal ion is selected in the group consisting of iron (Fe), copper (Cu), manganese (Mn), calcium (Ca), magnesium (Mg) and zinc (Zn). More advantageously, the metal ion is iron (Fe), copper (Cu), manganese (Mn), calcium (Ca), or mixtures thereof, preferably iron (Fe) and/or copper (Cu). More advantageously, the metal ion is iron (Fe), copper (Cu), manganese (Mn) or calcium (Ca), preferably iron (Fe) or copper (Cu).

In particular, the metal ion can be used as such or can be in the form of a metallic salt or in the form of a metallic complex, for example. For example, the metallic complex can be a coordination complex comprising the metal ion and a ligand.

In all these embodiments of the invention, the hyaluronate used may be a hyaluronic acid or any acceptable salt form of hyaluronic acid. As used herein, the term “hyaluronate” means hyaluronic acid and its equivalents, which includes hyaluronic acid of varying molecular weights and any of their salt forms, such as sodium hyaluronate.

Hyaluronic acid is a linear mucopolysaccharide comprised of alternating glucuronic acid and N-acetyl-glucosamine residues that interact with other proteoglycans to provide stability and elasticity to the extracellular matrix of all tissues. This network of macromolecules regulates tissue hydration and the movement of substances within the interstitial compartment.

Hyaluronic acid may be used in the form of an acid, or in the form of a hyaluronic acid salt. Suitable salt form includes, but is not limited to, an alkali metal salt such as a sodium or potassium salt, an alkaline earth metal salt such as a magnesium or calcium salt.

In particular, the hyaluronate (HA) have a molecular weight between 1 kDa and 10 000 kDa, preferably between 100 kDa and 10 000 kDa.

In a specific embodiment, the hyaluronate (HA) have a molecular weight between 200 kDa and 9000 kDa, or between 300 kDa and 8000 kDa, or between 400 kDa and 7000 kDa, or between 500 kDa and 6000 kDa, or between 600 kDa and 5000 kDa, or between 600 kDa and 4000 kDa, or between 600 kDa and 3000 kDa, or between 600 kDa and 2000 kDa, in particular between 600 kDa and 1000 kDa.

In a specific embodiment, the hyaluronate (HA) have a molecular weight between 600 kDa and 1000 kDa.

In particular, the hyaluronate can be used as such or in the form of a conjugate. According to the invention, a hyaluronate conjugate can be, for example, a conjugate of a hyaluronate with an anticancer agent, an oligosaccharide, a carrier, or a vector.

In the context of the invention, when the metal ion is in combination with hyaluronate or hyaluronate conjugate, the molar ratio metal ion/hyaluronate or hyaluronate conjugate can be comprise between 1:100 and 100:1, preferably 1:1.

In a particular aspect of the invention, the pharmaceutical composition can comprise transferrin instead of the metal ion or instead of the hyaluronate or hyaluronate conjugate.

The pharmaceutical compositions contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the metal and/or hyaluronate and that is not toxic to the host to which it is administered. In particular, the pharmaceutical composition is adapted for an intralymphatic administration. For example, the composition may be formulated in a unit dosage form for injection in vehicles such as saline, dextrose solution, serum albumin and Ringer's solution. The pharmaceutical composition can be formulated as solutions In pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for intralymphatic administration may be in the form of a solution or a suspension in an aqueous liquid or non-aqueous liquid; or in the form of an oil-in-water emulsion or a water-in-oil emulsion. Every such formulation can also contain other pharmaceutically compatible and nontoxic auxiliary agents, such as, e.g. stabilizers, antioxidants, binders, dyes, emulsifiers or flavoring substances. The formulations of the present invention comprise a metal ion, hyaluronate (HA), hyaluronate conjugate, or combination thereof in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection of suitable sterile solutions. Methods for the safe and effective administration directly into the lymphatic system are known to those skilled in the art.

In a specific aspect of the invention, the method for the treatment of cancer further comprises simultaneously, separately or sequentially administering at least one anticancer agent, in particular a known anticancer agent for chemotherapy, radiotherapy, surgery, or immunotherapies.

In particular, the method for the treatment of cancer further comprises simultaneously, separately or sequentially administering at least one anticancer agent for radiotherapy or chemotherapy, such as doxorubicin, carboplatin, oxalliplatin, taxol, olaparid/parp inhibitors, or agent for targeted therapies

In particular, the method for the treatment of cancer further comprises simultaneously, separately or sequentially administering at least one immuno-oncology agent.

In the context of the invention, immuno-oncology agents (also known as immuno-targeted agents) act against tumors, at least in part, by involving the immune system, or by an immune system-related mode of action. An immuno-oncology may more particularly act by modulating the action of immune cells.

Examples of immuno-oncology agents comprise agents that modulate immune checkpoints such as 2B4, 4-1BB (CD137), AaR, B7-H3, B7-H4, BAFFR, BTLA, CD2, CD7,CD25, CD27, CD28, CD30, CD40, CD80, CD83 ligand, CD86, CD160, CD200, CDS, CEACAM, CTLA-4, GITR, HVEM, ICAM-1, KIR, LAG-3, LAIR1, LFA-1 (CD 11 a/CD 18), LIGHT, NKG2C, NKp80, OX40, PD-1, PD-L1, PD-L2, SLAMF7, TGFRp, TIGIT, Tim3 and VISTA.

Immuno-oncology agents may be in the form of antibodies, peptides, small molecules or viruses. In a particular embodiment, the immuno-oncology agent is an antibody against PD-1, PD-L1 or PD-L2.

In a particular embodiment, the immuno-oncology agent is an inhibitor of arginase, CTLA-4, indoleamine 2,3-dioxygenase, CD25 and/or PD-1/PD-L1. In certain embodiments, the immuno-oncology agent is abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab, ozogamicin, intelumumab, ipilimumab, isatuximab, lambrolizumab, MED 14736, MPDL3280A, nivolumab, obinutuzumab, ocaratuzumab, ofatumumab, olatatumab, pembrolizumab, pidilizumab, rituximab, ticilimumab, samalizumab, or tremelimumab.

More generally, an immuno-oncology agent may be any agent that may be used in the treatment of malignant diseases and that acts, at least in part, by involving the immune system, or has an immune system-related mode of action. For example, the immuno-oncology agent may be selected from, without limitation:
an immune checkpoint inhibitor such as a PD-1 inhibitor, e.g. nivolumab or pembrolizumab;
an immune checkpoint inhibitor such as a PDL-1 inhibitor, e.g. atezolizumab, avelumab, or durvalumab; or a CTLA-4 inhibitor, e.g. ipilimumab,
a cancer vaccine, e.g. sipuleucel-T;
an immunomodulator such as thalidomide, lenalidomide, pomalidomide,
a non-specific immunotherapy, e.g. interferons, or interleukins; and
a chimeric antigen receptor (CAR)-T cell therapy, e.g. tisagenlecleucel, or axicabtagene ciloleucel, and
combinations thereof.

Advantageously, the method for the treatment of cancer further comprises simultaneously, separately or sequentially administering at least one PD-1/PD-L1 inhibitors.

In a specific aspect of the invention, the method for the treatment of immune deficiency diseases further comprises simultaneously, separately or sequentially administering at least one drugs that stimulate the immune system.

In a specific aspect of the invention, the method for the treatment of infectious diseases further comprises simultaneously, separately or sequentially administering at least one antibiotic agent.

The invention further relates to a method for treating cancer and/or immune deficiency diseases and/or infectious diseases in a subject in need thereof, comprising the intralymphatic administration to the subject of a therapeutically effective amount of the pharmaceutical composition according to the invention or the complex of the invention or the pharmaceutical combination of the invention.

The invention further relates to the use the pharmaceutical composition according to the invention or the complex of the invention or the pharmaceutical combination of the invention, for the manufacture of a medicament for use in a method for treating cancer and/or immune deficiency diseases and/or infectious diseases, wherein said method comprises intralymphatic administration of the pharmaceutical composition to a subject.

Further aspects and advantages of the present invention will be described in the following examples, which should be regarded as illustrative and not limiting.

### EXAMPLES

### Example 1

### Materials and Methods

### Cell Culture

Work was carried out using primary monocytes obtained from peripheral blood samples of distinct human donors from the EFS (Etablissement Français du Sang). Pan monocytes were isolated by negative magnetic sorting using microbeads according to the manufacturer's instructions (Miltenyi Biotec, 130-096-537), and cultured in RPMI 1640 supplemented with GlutaMAX (Gibco, 61870010), 10% fetal bovine serum (FBS, Eurobio Scientific, CVFSVF00-01). To generate monocyte-derived macrophages (MDM), pan monocytes were treated with granulocyte-macrophage colony-stimulating factor (GM-CSF, Miltenyi Biotec, 130-093-866, 100 ng/ml) for 5 days. Subsequently, lipopolysaccharide (LPS, InvivoGen, tlrl-3pelps, 100 ng/ml) and interferon-γ (IFNγ, Miltenyi Biotec, 130-096-484, 20 ng/ml) were added to the medium for 24 h to generate activated MDM (aMDM). CD4⁺ T cells were isolated from peripheral blood samples by negative magnetic sorting using microbeads according to the manufacturer's instructions (Miltenyi Biotec, 130-096-533) and cultured in RPMI 1640 supplemented with glutamine and 10% fetal bovine serum. Subsequently, CD3/CD28 antibodies were added to the medium for 48 h to generate activated CD 4⁺ T cells. CD 8⁺ T cells were isolated from peripheral blood samples by negative magnetic sorting using microbeads according to the manufacturer's instructions (Miltenyi Biotec, 130-096-495) and cultured in RPMI 1640 supplemented with glutamine and 10% fetal bovine serum. Subsequently, CD3/CD28 antibodies were added to the medium for 48 h to generate activated CD4⁺ T cells. To generate anti-inflammatory macrophages, pan monocytes were treated with macrophage colony-stimulating factor (M-CSF, Miltenyi Biotec, 130-096-492, 100 ng/ml) for 5 days and subsequently, IL-4 (Miltenyi Biotec, 130-093-921, 20 ng/ml) was added to the medium for 24 h. To generate dendritic cells (DC), pan monocytes were treated with GM-CSF (100 ng/ml) and IL-4 (10 ng/ml) for 5 days. Subsequently, LPS (100 ng/mL) was added to the medium to generate activated DC (aDC).

### Flow Cytometry

For human immune cells: cells were washed with ice-cold 1× PBS, incubated with Fc block (Human TruStain FcX, BioLegend, 422302, 1:20) for 15 min, subsequently incubated with antibodies for 20 min at 4° C. in 1× PBS/ 0.5 % bovine serum albumin (BSA) and then washed before analysis using a flow cytometer (BD LSRFortessa X-20). Cells were analyzed with the corresponding antibody panels.

### Inductively Coupled Plasma Mass Spectrometry (ICP-MS)

Glass vials equipped with Teflon septa were cleaned with nitric acid 65% (VWR, Suprapur, 1.00441.0250), washed with ultrapure water (Sigma-Aldrich, 1012620500) and dried. Cells were harvested and washed twice with 1× PBS. Cells were then counted using an automated cell counter (Entek) and transferred in 200 μL 1× PBS or ultrapure water to the cleaned glass vials. The same volume of 1× PBS or ultrapure water was transferred into separate vials for the background subtraction, at least in duplicate per experiment. Samples were lyophilized using a freeze dryer (CHRIST, 2-4 LDplus). Samples were subsequently mixed with nitric acid 65% and heated at 80° C. overnight in the same glass vials closed with a lid carrying a Teflon septum. Samples were then cooled to room temperature and diluted with ultrapure water to a final concentration of 0.475 N nitric acid and transferred to metal-free centrifuge vials (VWR, 89049-172) for subsequent mass spectrometry analyses. Amounts of metals were measured using an Agilent 7900 ICP-QMS in low-resolution mode, taking natural isotope distribution into account. Sample introduction was achieved with a micro-nebulizer (MicroMist, 0.2 mL/min) through a Scott spray chamber. Isotopes were measured using a collision-reaction interface with helium gas (5 mL/min) to remove polyatomic interferences. Scandium and indium internal standards were injected after inline mixing with the samples to control the absence of signal drift and matrix effects. A mix of certified standards was measured at concentrations spanning those of the samples to convert count measurements to concentrations in the solution. Values were normalized against cell number, dry weight or volume.

### RNA Interference

Human primary monocytes were transfected with Human Monocyte Nucleofector kit (Lonza, VPA-1007) according to the manufacturer's instructions. 5×10⁶ monocytes were resuspended in 100 □L nucleofector solution with 200 pmol of ON-TARGETplus SMARTpool siRNA or negative control siRNA (Qiagen, 1027310) before nucleofection with Nucleofector II (Lonza). Cells were then immediately removed and incubated overnight with 5 mL of prewarmed complete RPMI medium (Gibco). The following day, GM-CSF was added to the medium. The target genes used are as follows:
Target gene: CD44 (protein: CD44);
Target gene: SLC31A1 (protein: Ctr1)
Target gene: SLC31A2 (protein: Ctr2)
Target gene: SLC11A2 (protein: DMT1)
Target gene: SLC25A3 (protein: SLC25A3)
Target gene: SLC25A37 (protein: SLC25A37/Mitoferrin-1)
Target gene: TFRC (protein: TfR1)

### Genome Editing

CRISPR knockout (ko) was performed using the following strategy. Human primary monocytes were transfected with Human Monocyte Nucleofector kit (Lonza, VPA-1007). 5×10⁶ monocytes were resuspended in 100 □L nucleofector solution with a 100 pmol CAS9 (Dharmacon, CAS12206)/200 pmol CD44 (Dharmacon, SQ-009999-01-0010) sgRNA mix. The CAS9/CD44 mix was incubated for 10 min at 37° C. before nucleofection with Nucleofector II (Lonza). Cells were then immediately removed and incubated overnight with 5 mL of prewarmed complete RPMI medium (Gibco) and differentiated into dendritic cells as detailed above. The following sequences were used: CD44 sgRNA ((Edit-R Human Synthetic CD44, set of 3, target sequences)

### Antibodies

FC: flow cytometry, Hu: used for human samples. CD86-PE/Cy7 (BD Biosciences, 561128, clone 2331 (FUN-1), Lot 1309531, FC 1:33, Hu), anti-human CD44 therapeutic antibody (RG7356, Creative Biolabs, TAB-128CL, 10 □g/mL), CD44-AF647 (Novus Biologicals, NB500-481AF647, clone MEM- 263, Lot 118753, FC 1:100, Hu), CD40-BV510 (BioLegend, 334329, clone 5C 3, Lot 312131, FC 1:100, Hu), CD80-AF700 (BD Biosciences, 561133, clone 307.4, Lot 1060235, FC 1:100, Hu), CD83-PE (BioLegend, 305307, clone HB 15e, Lot B303073, FC 1:100, Hu), CD25-BV711 (BioLegend, 302636, clone BC96, Lot B281779, FC 1:100, Hu), CD69-PerCP (BioLegend, 310928, clone FN50, Lot B290414, FC 1:100, Hu), CD14-Krome Orange (Beckman Coulter, B01175, clone RMO/ 52, Lot 200040, FC 1:100, Hu), CD16-Pacific Blue (Beckman Coulter, B36292, clone 3G 8, Lot 200029, FC 1:100, Hu), CD163-PE (BD Biosciences, 556018, clone GHI/ 61, Lot 9143793, FC 1:100, Hu), CD86-PE (BioLegend, 105007).

### Animals

Balb/C mice (25 weeks adult females) were purchased from Charles River and grow in CRCM animal core facility. Mice were housed under sterile conditions with sterilized food and water provided ad libitum and maintained on a 12-h light and 12-h dark cycle. Mice were not subjected to any procedures prior to the lymph, blood and serum samples collection. Animal experiments were performed in accordance with the European Community guiding in the care and use of animals.

### Lymph Sample Collection

Thirty minutes before the beginning of experiment, buprenorphine (Buprecare), an analgesic, was administered by intraperitoneal injection (0.5 mg/kg). Mice were anesthetized by lethal intraperitoneal injection of ketamine/xylazine combination. After cutaneus and peritoneal incisons, the lymph collection was performed from the intestinal lymph trunk (MIHAI IONAC, 2003, DOI: 23: 239-245) with TransferTrip (Eppendorf, ref 930001040), frozen at −20° C. and stocked at −80° C.

### Blood and Serum Sample Collection

After lymph collection, we performed a terminal cardiac puncture (23 G needle with 1 to 2ml syringe) with thoracotomy to collect a large volume of blood without anticoagulants. 100-200 μl of blood sample are placed in a vial, frozen at −20° C. and stocked at −80° C. The rest of blood collection was left at room temperature for 30 minutes, then centrifuged at 2000× g for 15 minutes. The supernatant (serum) was collected in a vial, frozen at −20° C. and stocked at −80° C.

### Tumor Models

### Animals

C57Bl/6 or Balb/C mice (6-10 weeks adult females) were purchased from Charles River or Janvier and kept in the CRCM animal core facility. Mice were housed under sterile conditions with sterilized food and water provided ad libitum and maintained on a 12-h light and 12-h dark cycle. Mice were not subjected to any procedures prior to the lymph, blood and serum sample collection. Animal experiments were performed in accordance with the European Community Guidance for care and use of animals.

### Breast Cancer Cells Graft With Lymph Collection

4T1 or EO71 cells were inoculated in the mammary fat pad with suspended in 50% phenol red-free Matrigel (Becton Dickinson Bioscience). Tumor growth was monitored by measuring with a digital caliper and calculating tumor volume (length×width²×π/6). When the tumor is found, a lymph sample will be collected.

### Breast Cancer Cells Graft With Injection in Inguinal Ganglion

4T1 or EO71 cells were inoculated in the mammary fat pad with suspended in 50% phenol red-free Matrigel (Becton Dickinson Bioscience). Tumor growth was monitored by measuring with a digital caliper and calculating tumor volume (length×width²×π/6). 10 μl of solution was injected into the inguinal ganglion draining the tumor.

### Results

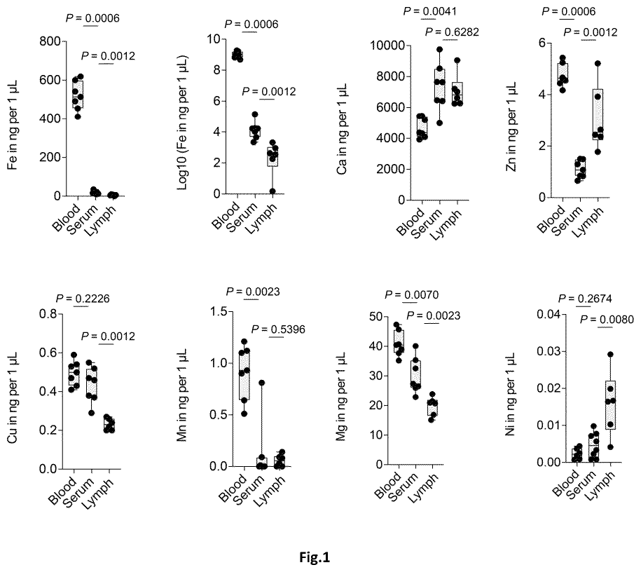

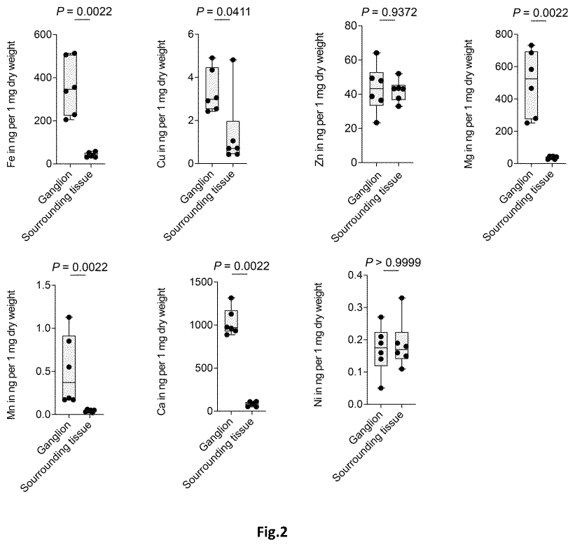

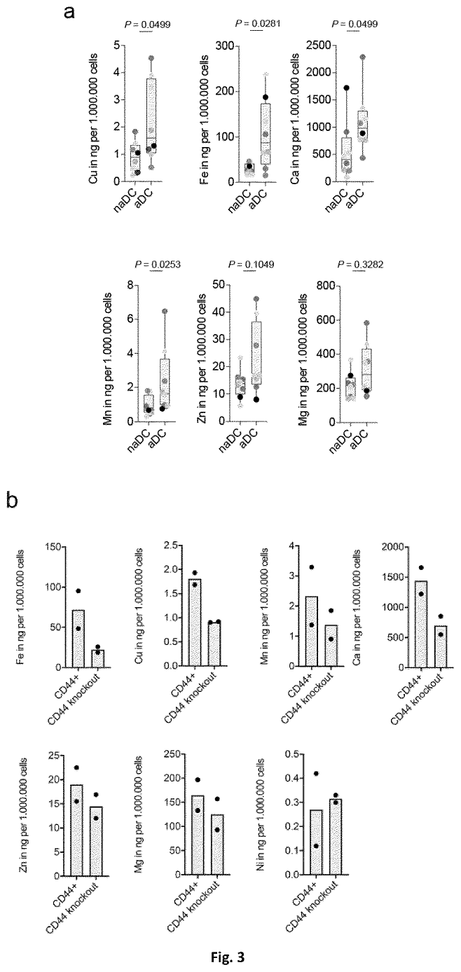

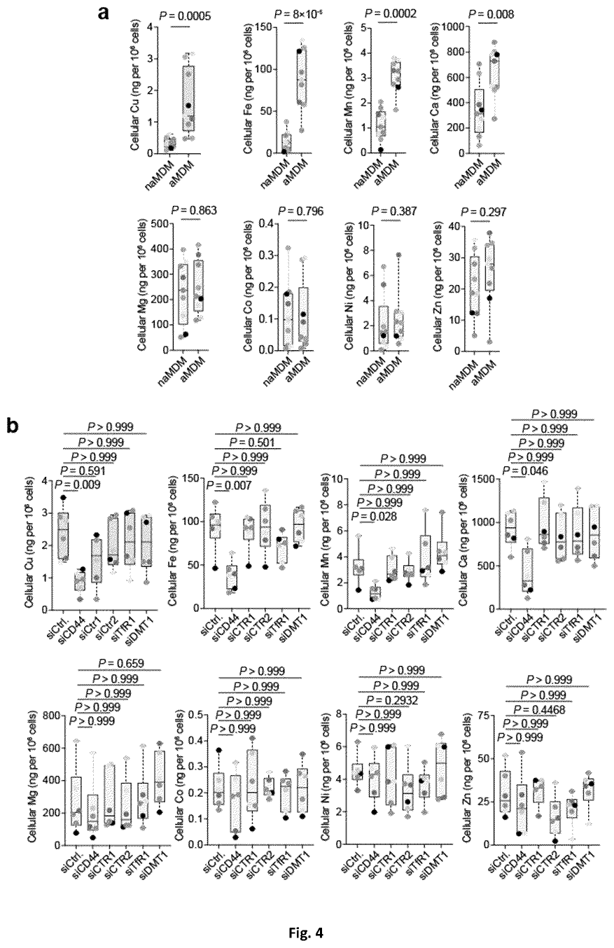

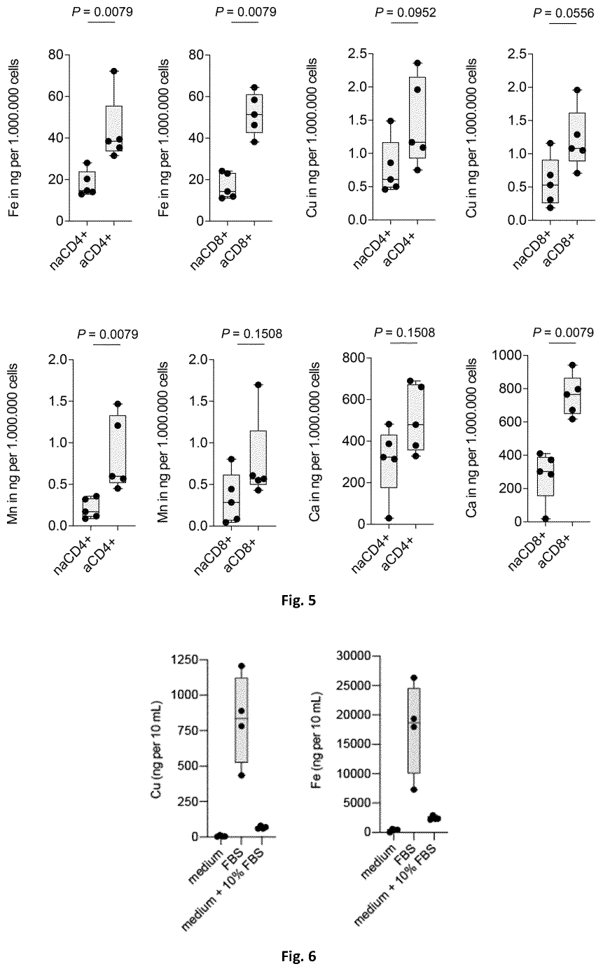

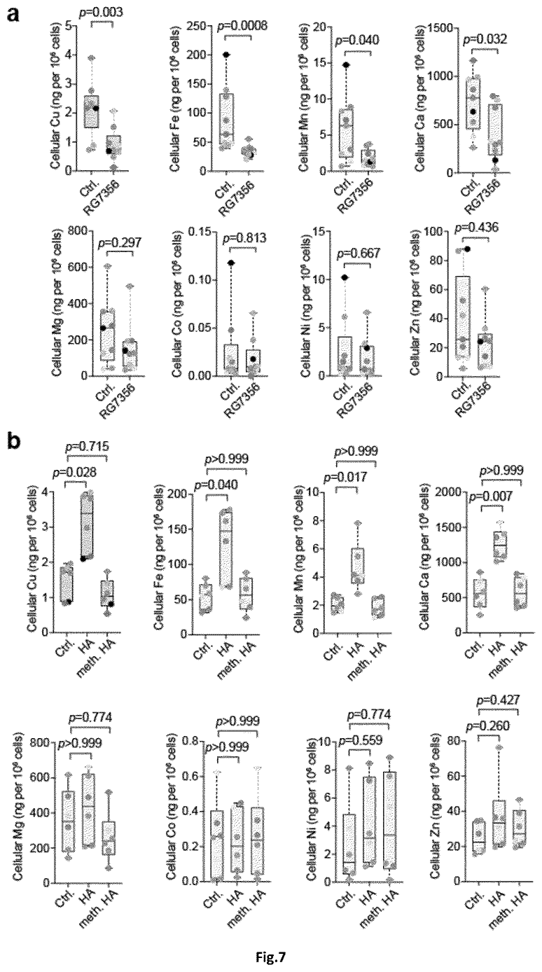

The inventors have shown that the lymphatic system is deprived of metals (see FIGS. 1 to 7), and concentrations vary to the blood. Metal concentrations are different in the ganglion compared to the surrounding tissue.

The invention thus describes the use of metals and hyaluronates supplemented directly in the lymphatic system to enable immune cells activation. Hyaluronates and metals (iron, copper, manganese . . . ) boost immune cell activation and anti-cancer activity when the treatment is supplied directly in the lymphatic system, in particular via the lymph node. Metal iron overload, such as iron, can lead to ferroptosis in cancer cells, and in particular the persister cancer state and circulating tumor cells (see Nature: Nat Chem. 2020 October; 12(10): 929-938. doi: 10.1038/s 41557-020-0513-5). Thus, the strategy proposed herein can activate the immune system whilst inducing ferroptosis in cancer cells.

### EXAMPLE 2

### Hyaluronan Metal Complex Composition and Experimentation

Hyaluronate (HA) of an average size of 600-1000 kDa (Biosynth, FH71634). Using an average size of 800 kDa, molarity was calculated for a final concentration of 1 mg/mL HA. Then, a molarity of 50% of Hyal was calculated to give the final molarity of metals to be added. CuCl₂, MnCl₂ and FeC₃ were added in this order in a 10:10:80 ratio. The final solution was adjusted with NaCl to obtain a 0.9% final saline solution, and sterile filtered. 10 μL of this solution was injected into the lymphatic system of mice.

### Ethics Statement

For mouse lymph and blood collection, animal experiments were performed in accordance with the European Community guiding in the care and use of animals. Animal experiments were performed in agreement with the French Guidelines for animal handling and approved by local ethics committee (Agreement no. 16487-2018082108541206 v3).

### Isolation of Blood, Serum and Lymphatic Fluid From Mice

Balb/C mice (25-week-old adult female mice) were purchased from Charles River and grow in CRCM animal core facility. Mice were housed under sterile conditions with sterilised food and water provided ad libitum and maintained on a 12 h light/dark cycle. Mice were not subjected to any procedures prior to the lymph, blood and serum samples collection. Lymph sample collection: Thirty min before the beginning of the experiment, buprenorphine (Buprecare), an analgesic, was administered by intraperitoneal injection (0.5 mg/kg). Mice were euthanised by intraperitoneal injection of a ketamine/xylazine combination (ketamine 100 mg/kg (Imalgène)/xylazine 10 mg/kg (Rompum); 20 μl/g). After cutaneous and peritoneal incisions, the lymph has been collected in the intestinal lymph trunk with a glass capillary. The lymph collection was placed in cryotubes, frozen at −20° C. and stocked at −80° C. Blood and serum sample collection: After lymph collection, we performed a terminal cardiac puncture (23 G needle with 1 to 2 ml syringe) with thoracotomy to collect a large volume of blood without anticoagulants. 100 to 200 μL of blood sample were placed in a vial, frozen at −20° C. and stocked at −80° C. The rest of the blood collection was left to stand at room temperature for 30 min, then centrifuged at 2000×g for 15 min. The supernatant (serum) was collected in a vial, frozen at −20° C. and stored at −80° C.

### Intranodal Murine Metastasis Models and Hyaluronan-metal Complex Treatment

Murine breast cancer cells (4T1) were transplanted into 6 to 8-week-old female Balb/c mice (syngeneic with the 4T1 model). To perform injections into lymph nodes, the lymphatics were first traced by injecting 2% Evans Blue dye (Sigma-Aldrich, E2129,) into the foot pedal 5 min before performing intranodal injections. After injecting Evans Blue dye, the mice were anesthetised using isoflurane and a small (5-10 mm) incision was made in the region of the right popliteal lymph node. The lymph node was located based on Evans Blue staining, immobilised with forceps, and 20000 cells (Experiment 1) to 10000 cells (Experiments 2 and 3) suspended in 1× PBS were injected in a volume of 10 μL into the popliteal lymph node using a 27 G Hamilton syringe. Injection into the lymph node was confirmed by visible swelling of the lymph node. The incision was closed using surgical glue (3M VetBond Tissue Adhesive, 1469SB,) and the mice were closely monitored for signs of pain or distress. Once tumours were palpable in at least 75% of the mice (~1 week after injection), 10 μL of hyaluronan-metal complex or of vehicle delivered intralymphatically into the tumour-bearing lymph node every other day until the experimental endpoint. At that point, all mice in the cohort were killed, per approved protocol, for analysis of intranodal tumour diameter, tumour mass and mouse. Tumour samples were frozen in 10% DMSO in FBS (1° C./min until −80° C.) for subsequent cellular analyses. No formal randomisation techniques were used. However, animals were allocated randomly to treatment groups and specimens were processed in an arbitrary order. For all experiments, the maximum permitted tumour diameter was 2.0 cm and this limit was not exceeded in any experiment. For all experiments, mice were kept on a normal Chow diet and fed ad-libitum.

### Dissociation of Murine Tumour Samples

Mouse tumour samples were dissociated using the mouse tumour dissociation kit (Miltenyi, 130-096-730) according to the manufacturer's protocol. Tumours were cut into small pieces of 1-5 mm, put in presence of the enzyme mix in RPMI and dissociated on the gentleMACS Octo Dissociator with Heaters (Miltenyi) with the appropriate gentleMACS program (37C_m_TDK). Subsequently, the dissociated tumour suspension was applied to a MACS SmartStrainer (30 μm) (Miltenyi). Samples were diluted with 1× PBS (Phosphate-buffered saline) and centrifuged at 300×g. The cell pellet was resuspended in RPMI (10% FBS, penicillin/streptomycin) and cells were counted using an automated cell counter (Entek).

### Flow Cytometry

Cells were washed with ice-cold 1× PBS. For antibody staining, cells were incubated with Fc block (Human TruStain FcX, Biolegend, 422302, 1:20) for 15 min, then incubated with antibodies in ice cold 10% FBS, 1× PBS, 2 mM EDTA for 20 min at 4° C. and then washed with 1× PBS and resuspended in 10% FBS, 1× PBS, 2 mM EDTA before analysis using a flow cytometer. Data were recorded on an Flow. All data were analysed with FlowJo software v. Relevant antibodies with fluorophores were used for the respective multi-panel staining.

### Results

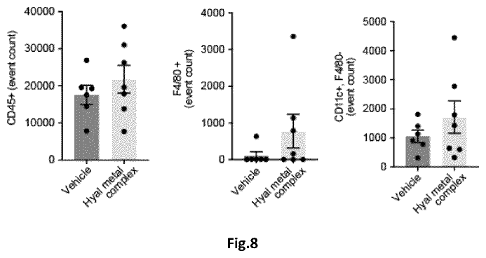

The Results Are Summarized in FIG. 8.

These results show that metal/hyaluronate complexes have an effect on tumors in mice in vivo. They change the immune infiltrate of tumors increasing the amount of total myeloid cells present in the tumor. In particular, increase of F4/80³⁰ cells indicate an increased infiltration of macrophages and an increase of CD11⁺, F4/80⁻ indicates an increase of dendritic cells in the tumors.

Sources of funding: European Research Council under the European Union's Horizon 2020 research and innovation programme (grant agreement no. 647973), Foundation Charles Defforey-Institut de France, Ligue Contre le Cancer

1-18. (canceled)

19. A method of treating cancer and/or immune deficiency diseases and/or infectious diseases comprising administering a pharmaceutical composition comprising a metal ion and optionally hyaluronate (HA) or a hyaluronate conjugate via intralymphatic administration of the pharmaceutical composition to a subject, wherein the metal ion is selected from the group consisting of iron (Fe), copper (Cu), manganese (Mn), calcium (Ca), magnesium (Mg), zinc (Zn), and mixtures thereof.

20. The method according to claim 19, wherein the method comprises the injection of the pharmaceutical composition into the lymphatic system, a lymph vessel, or a lymph node.

21. The method according to claim 19, wherein the metal ion is selected from the group consisting of iron (Fe), copper (Cu), manganese (Mn), calcium (Ca), and mixtures thereof.

22. The method according to claim 19, wherein the metal ion is iron (Fe) or copper (Cu), or mixtures thereof.

23. The method according to claim 19, wherein the pharmaceutical composition comprises a combination of a metal ion and hyaluronate (HA) or a hyaluronate conjugate.

24. The method according to claim 19, wherein the hyaluronate (HA) have a molecular weight between 1 and 10,000 kDa.

25. The method according to claim 19, said method further comprising simultaneously, separately or sequentially administering at least one immuno-oncology agent.

26. A method for the treatment of cancer and/or immune deficiency diseases and/or infectious diseases in a subject, comprising intralymphatic administration of a complex of hyaluronate or hyaluronate conjugate and at least one metal ion or a pharmaceutical composition comprising said complex and a pharmaceutically acceptable excipient and/or carrier, to the subject, wherein said hyaluronate presents a molecular weight between 1-10,000 kDa and said metal ion being selected from the group consisting of iron (Fe), copper (Cu), manganese (Mn), calcium (Ca), magnesium (Mg), zinc (Zn), and mixtures thereof.

27. The method according to claim 26, said method further comprising simultaneously, separately or sequentially administering at least one immuno-oncology agent.

28. A method for the treatment of cancer and/or immune deficiency diseases and/or infectious diseases comprising the administration of a pharmaceutical combination to a subject in need of treatment, said pharmaceutical combination comprising:
(i) at least one metal ion selected from the group consisting of iron (Fe), copper (Cu), manganese (Mn), calcium (Ca), magnesium (Mg), zinc (Zn), and mixtures thereof; and
(ii) hyaluronate (HA) or a hyaluronate conjugate,
and wherein said method comprises the simultaneous, separate or sequential administration of (i) and (ii) directly into the lymphatic system, a lymph node or a lymph vessel.

29. The method according to claim 28, wherein the pharmaceutical combination is a composition comprising (i), (ii) and a pharmaceutically acceptable carrier, or the pharmaceutical combination is a kit of parts comprising (i) and (ii).
